(19) United States
(12) Patent Application Publication
(10) Pub. No.: US 2026/0275389 A1
(43) Pub. Date: Sep. 17, 2026

(54) USE OF TYPE III POLYKETIDE SYNTHASES FROM ASCOMYCETE FUNGI AS PHLOROGLUCINOL SYNTHASES

(71) Applicant: COMPAGNIE GENERALE DES ETABLISSEMENTS MICHELIN, Clermont-Ferrand (FR)

(72) Inventors: GOVINDAN RAGHUNATHAN, Toulouse (FR); NICOLAS CANU, Toulouse (FR); MOHAMMED EL BACHIRI, Toulouse (FR); RAJENDRA SHARMA, Toulouse (FR); GIULIANO NIGRO, Saint Laurent Du Var (FR); NICOLAS PANEL, Toulouse (FR); SOPHIE BOZONNET, Toulouse (FR); MAGALI REMAUD-SIMEON, Ramonville (FR); VINCENT LAFAQUIERE, Clermont-Ferrand (FR); ISABELLE ANDRE, Toulouse (FR)

(21) Appl. No.: 19/469,914

(22) PCT Filed: Mar. 28, 2024
(86) PCT No.: PCT/FR2024/050406

§ 371 Date: Sep. 26, 2025

(30) Foreign Application Priority Data
Mar. 28, 2023 (FR) FR2302963

(51) Int. Cl. C12P 7/22; C12N 9/10

(52) U.S. Cl. CPC C12P 7/22; C12N 9/1029

(57) ABSTRACT

A polypeptide chosen from type III polyketide synthases from ascomycete fungi is used for producing phloroglucinol. Also disclosed is a nucleic acid molecule encoding same, a vector comprising a nucleic acid molecule encoding same and a host cell expressing same Also disclosed are particular nucleic acid molecules encoding a cyanobacterial type III polyketide synthase, vectors and host cells, and also methods for producing phloroglucinol.

### FIELD OF THE INVENTION

The present invention lies in the fields of microbial biochemistry and more particularly in the field of microbial enzymatic synthesis of phloroglucinol. The invention relates to the use of type III polyketide synthases from ascomycete fungi as phloroglucinol synthases, to associated phloroglucinol production methods, and also to nucleic acids, vectors and host cells intended for phloroglucinol production.

### PRIOR ART

Phloroglucinol is an aromatic organic compound used notably in the manufacture of pharmaceutical products and explosives.

Phloroglucinol synthesis is catalysed by certain type III polyketide synthases known as phloroglucinol synthases. Phloroglucinol synthases perform the condensation of three malonyl-CoA molecules to form one phloroglucinol molecule, according to the following scheme (Reaction I):

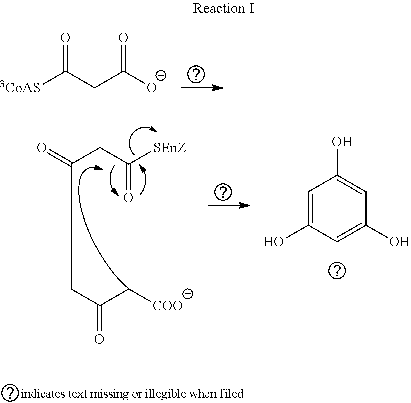

Many oligomers can then be synthesized from phloroglucinol, such as phlorotannins. Phlorotannins notably include fucols, phloretols and fucophloretols, which are derivatives of phloroglucinol making up the wall of brown algae. Various protective activities of brown algae have also been attributed to phlorotannins.

The natural synthesis of phloroglucinol was initially described in the Gram-negative bacterium Pseudomonas fluorescens (Achkar et al., 2005; Zha et al., 2006) and in the brown alga Ectocarpus siliculosus (Meslet-Cladière et al., 2013). The enzyme phloroglucinol synthase involved in phloroglucinol synthesis was identified in both these species.

In Pseudomonas fluorescens, phloroglucinol synthase is encoded by the PHLD gene (Achkar et al., 2005; Zha et al., 2006). The phloroglucinol synthase activity of PHLD was demonstrated in Escherichia coli expressing a heterologous PHLD gene (Achkar et al., 2005). This activity was confirmed in vitro, by small-scale enzymatic tests performed with a recombinant PHLD expressed and purified from Escherichia coli cultures (Zha et al., 2006).

In Ectocarpus siliculosus, phloroglucinol synthase is encoded by the PKS1 gene (Meslet-Cladière et al., 2013). The phloroglucinol synthase activity of PKS1 was demonstrated in vitro, from recombinant PKS1 expressed and purified in Escherichia coli and from cell extracts of E. siliculosus (Meslet-Cladière et al., 2013, WO2013/045510).

However, the PHLD and PKS1 enzymes have low enzymatic activity. Furthermore, it has been shown that the PHLD enzyme from Pseudomonas fluorescens and the PKS1 enzyme from Ectocarpus siliculosus produce little or no phloroglucinol when these sequences are expressed in yeast instead of Escherichia coli (WO2019/002799; WO2019/002798). However, eukaryotic systems may be advantageous, notably for large-scale production. Specifically, they allow the production of enzymes that can be modified at the post-translational level. WO2019/002799 and WO2019/002798 describe phloroglucinol synthases that are capable of synthesizing phloroglucinol at a much higher level than PKS1 from Ectocarpus siliculosus when sequences encoding these enzymes are transfected into yeast. These phloroglucinol synthases are derived from actinomycete bacteria (WO2019/002799) or eukaryotic algae (WO2019/002798).

There is however a need for other phloroglucinol synthases that are capable of synthesizing phloroglucinol at high levels, notably when sequences encoding these enzymes are transfected into a host cell, in particular into yeast.

However, type III polyketide synthases are a broad class of enzymes collating proteins with sequence similarities while at the same time displaying very dissimilar enzymatic activities (Meslet-Cladière et al., 2013). Thus, phloroglucinol synthase activity is just one of the multiple activities likely to be present in a type III polyketide synthase, and only a small proportion of type III polyketide synthases thus have phloroglucinol synthase activity. In addition, no sequence unit associated with phloroglucinol synthase activity has been described, making the identification of type III polyketide synthases having such activity particularly difficult.

### DESCRIPTION OF THE INVENTION

In a surprising manner, since genes encoding phloroglucinol synthases had hitherto only been identified in bacteria and algae, the Inventors were however able to identify new phloroglucinol synthases from ascomycete fungi, which are capable of synthesizing phloroglucinol at an advantageous level when sequences encoding these enzymes are transfected into a host cell, in particular into a yeast or a bacterium.

Thus, according to a first aspect, the invention relates to the use of a polypeptide chosen from type III polyketide synthases from ascomycete fungi, to a nucleic acid molecule encoding same, to a vector comprising a nucleic acid molecule encoding same or to a host cell expressing same, for producing phloroglucinol.

According to a second aspect, the invention relates to an isolated nucleic acid molecule comprising a nucleic acid sequence encoding a polypeptide as defined in the use according to the invention, characterized in that:
a) the isolated nucleic acid molecule also comprises a promoter controlling the expression of the nucleic acid sequence; or
b) the isolated nucleic acid molecule also comprises a transcription terminator controlling the expression of the nucleic acid sequence; or
c) the nucleic acid sequence is also optimized for expression in a host cell, in particular in a yeast or in a bacterium; or
d) any combination of a) to c).

According to a third aspect, the invention relates to a vector comprising a nucleic acid molecule according to the invention, said vector preferably being a plasmid.

According to a fourth aspect, the invention relates to a host cell comprising a nucleic acid molecule according to the invention or a vector according to the invention.

According to a fifth aspect, the invention relates to a method for producing phloroglucinol, comprising the steps consisting in:
(i) placing a host cell expressing the polypeptide chosen from type III polyketide synthases from ascomycete fungi as defined in the use according to the invention, for example a host cell according to the invention, in contact with a suitable substrate;
(ii) in vitro culturing of the host cell from step (i) under conditions allowing growth of said host cell and/or expression of the nucleic acid molecule contained in said host cell, so as to produce phloroglucinol;
(iii) optionally, recovering the phloroglucinol-comprising culture medium obtained after step (ii); and
(iv) optionally, purifying phloroglucinol from the culture medium of step (iii).

According to a sixth aspect, the invention relates to a method for producing phloroglucinol, comprising the steps consisting in:
(i) placing a polypeptide chosen from type III polyketide synthases from ascomycete fungi as defined in the use according to the invention in contact with malonyl-CoA;
(ii) incubating the mixture from step (i) under conditions suitable for producing phloroglucinol;
(iii) optionally recovering the phloroglucinol-comprising reaction medium obtained after step (ii); and
(iv) optionally, purifying phloroglucinol from the reaction medium of step (iii).

### DESCRIPTION OF THE FIGURES

FIG. 1 represents the alignment of the amino acid sequences SEQ ID NO: 1 (PhlD from Hirsutella minnesotensis 3608 designated PlhD-Hm2), SEQ ID NO: 2 (PhlD from Hirsutella minnesotensis 3608 designated PlhD-Hm1) and SEQ ID NO: 3 (PhlD from Aspergillus tanneri designated PlhD-At).

FIG. 2 represents the PBIM5 plasmid map used in the examples.

### DETAILED DESCRIPTION OF THE INVENTION

### Definitions

The term “a” or “an” means one or more. In other words, when “a” or “an” is used in relation to a feature, it covers both embodiments with the feature of interest only once, and those with the feature of interest several times. In other words, unless otherwise indicated (such as “only one” or “a single”), “a(n)” is used synonymously with “one or more” or “at least one”.

When used herein to define products, compositions and methods, the terms “comprising” (and any form of “comprising”, such as “comprises”), “having” (and any form of “having”, such as “has”), “including” (and any form of inclusion, such as “includes”) or “containing” (and any form of “containing”, such as “contains”) are open-ended and do not exclude additional, unmentioned elements or method steps. Thus, a polypeptide “comprises” an amino acid sequence when the amino acid sequence is part of the final amino acid sequence of the polypeptide. Such a polypeptide may have up to several hundred additional amino acid residues. The term “consisting essentially of” or “constituted essentially of” means the exclusion of other components or steps of any essential importance. Thus, a polypeptide “consists essentially of” an amino acid sequence when such an amino acid sequence is present with optionally only a few additional amino acid residues (for example, a peptide of not more than 20 amino acids, such as a label of 6 Hisx6 histidines, may additionally be present). “Consisting of” or “constituted of” means excluding more than traces of other components or steps. For example, a polypeptide “consists of” an amino acid sequence when the polypeptide does not contain any amino acids other than the indicated amino acid sequence.

The term “type III polyketide synthase” means a multifunctional enzyme or enzyme complex that produces polyketides and does not use an acyl carrier protein (ACP) domain.

The term “polyketide” means a large family of secondary metabolites in bacteria, mycetes, plants and certain animal lines, derived from the iterative condensation of acetyl or malonyl subunits by polyketide synthase enzymes. Polyketides also serve as starting materials for the manufacture of a wide range of natural and semi-synthetic products.

The term “phloroglucinol” means an aromatc organic benzene-1,3,5-triol compound having the following chemical formula (Formula I):

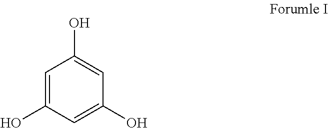

The term “phloroglucinol synthase” means a multifunctional enzyme or enzyme complex belonging to the type III polyketide synthase family, which catalyses the synthesis of phloroglucinol. A phloroglucinol synthase catalyses the condensation of three malonyl-CoA molecules to form one phloroglucinol molecule.

The term “enzymatic activity” or “catalytic activity” or even “activity” of an enzyme means the efficacy of an enzyme in converting a substrate into a product in a given medium. The term “enzyme efficacy” takes into account here the rate at which the enzyme converts the substrate into a product, and the degree of conversion of the substrate into a product by the enzyme. The term “degree of conversion of substrate into product by the enzyme” means here the ratio between the amount of end product obtained relative to the initial amount of substrate for a defined amount of enzyme. For the purposes of the invention, for example, enzymatic activity may be expressed as the amount of phloroglucinol produced in a given volume (in g/L).

The term “fungus” means a unicellular or multicellular organism of the Fungi kingdom. This kingdom comprises several divisions, including the Ascomycota, also known as higher fungi, i.e. fungi with a partitioned mycelium. The Ascomycota division comprises several subdivisions, including the Pezizomycotina, comprising filamentous fungi. This subdivision notably comprises the families Ophiocordycipitaceae (which notably includes the genus Hirsutella) and Trichocomaceae (which notably includes the genus Aspergillus). The genus Hirsutella notably comprises the species Hirsutella minnesotensis 3608. The genus Aspergillus notably comprises the species Aspergillus tanneri, Aspergillus aculeatus (notably the strains ATCC 16872/CBS 172.66/WB 5094), Aspergillus arachidicola, Aspergillus awamori, Aspergillus bertholletiae, Aspergillus bombycis, Aspergillus brasiliensis (notably the strains CBS 101740/IMI 381727/IBT 21946), Aspergillus caelatus, Aspergillus calidoustus, Aspergillus homomorphus (notably the strain CBS 101889), Aspergillus leporis, Aspergillus luchuensis (notably the strain CBS 106.47), Aspergillus miniscierotigenes, Aspergillus niger (notably the strains ATCC 1015/CBS 113.46/FGSC A1144/LSHB Ac4/NCTC 3858a/NRRL 328/USDA 3528.7, CBS 513.88/FGSC A1513), Aspergillus nomiae NRRL 13137, Aspergillus novoparasiticus, Aspergillus oryzae (notably the strains ATCC 42149/RIB 40), Aspergillus parasiticus (notably the strains ATCC 56775/NRRL 5862/SRRC 143/SU-1), Aspergillus pseudonomiae, Aspergillus pseudotamarii, Aspergillus steynii IBT 23096, Aspergillus tamarii, Aspergillus uvarum CBS 121591, Aspergillus violaceofuscus (notably the strain CBS 115571), and Aspergillus welwitschiae. The Ascomycota division also comprises the Taphrinomycotina and Saccharomycotina subdivisions. The subdivision Saccharomycotina comprises the order Saccharomycetales (budding yeasts), which comprises the genera Saccharomyces (notably including the species Saccharomyces cerevisiae, Saccharomyces boulardii, Saccharomyces douglasii and Saccharomyces bayanus), Candida, Eremothecium, Dekkera (notably including the species Dekkera brucelensis and Dekkera intermedia), Pichia (Hansenula), Debaryomyces, Lodderomyces, Yarrowia, Zigosaccharomyces (notably including the species Zigosaccharomyces bailii), Torulaspora (notably including the species Torulaspora globosa and Torulaspora glabrata), Kluyveromyces (notably including the species Kluyveromyces themotolerens), and Brettanomycces (notably including the species Brettanomycces custersii and Brettanomycces intermedius). The subdivision Taphrinomycotina comprises the class Schizosaccharomycetes (yeasts which reproduce by splitting, which notably comprises the genus Schizosaccharomyces which notably includes the species Schizosaccharomyces pombe). The Fungi kingdom also comprises the division Basidiomycota, which comprises the genera of Cryptococcus and Malassezia yeasts.

The term “PHLD.Pf” means either the gene encoding the Pseudomonas fluorescens PHLD phloroglucinol synthase, or the polypeptide encoded by this gene.

The term “PKS1.Es” or “PHLD.Es” means either the gene encoding the Ectocarpus siliculosus PKS1 phloroglucinol synthase, or the polypeptide encoded by this gene.

The term “PhlD” or “PHLD” denotes herein a candidate gene encoding a candidate phloroglucinol synthase enzyme, or the polypeptide encoded by this gene. According to the nomenclature chosen by the Inventors, “PhlD.ii” or “PHLD.ii” here denotes the candidate gene or candidate polypeptide from a given organism. The letters “ii” represent the initials of the genus and species to which said organism belongs. For example, PhlD.Hm corresponds to a candidate phloroglucinol synthase enzyme from a Hirsutella minnesotensis species, and PhlD.At corresponds to a candidate phloroglucinol synthase enzyme from an Aspergillus tanneri species. A number may follow the initials when several candidate phloroglucinol synthase enzymes have been identified in the same species.

The term “apolar amino acids” or “nonpolar amino acids” means a family of amino acids in which the average positions of the positive and negative partial charges are merged. Of the 20 conventional amino acids, the apolar amino acids are: glycine (G), alanine (A), valine (V), leucine (L), isoleucine (I), proline (P), tryptophan (W), phenylalanine (F), tyrosine (Y), methionine (M) and cysteine (C). Those other than glycine (G) can be subdivided into three sub-families: 1) “aliphatic apolar amino acids”, which have an aliphatic-type side chain and include alanine (A), valine (V), leucine (L), isoleucine (I), and proline (P), 2) “aromatic apolar amino acids”, which have an aromatic-type side chain and include tryptophan (W), phenylalanine (F), and tyrosine (Y), and 3) “sulfur-based apolar amino acids” whose side chain comprises a sulfur atom and which include methionine (M) and cysteine (C).

The term “polar amino acids” means a family of amino acids in which the average positions of the positive and negative partial charges are not merged. Among the 20 conventional amino acids, the polar amino acids are: serine (S), threonine (T), asparagine (N), glutamine (Q), glutamic acid (E), aspartic acid (D), lysine (K), arginine (R), histidine (H). They may be subdivided into three sub-families: 1) “neutral (or uncharged) polar amino acids”, which have an uncharged side chain and include serine (S), threonine (T), asparagine (N), and glutamine (Q), 2) “negatively charged polar amino acids”, which have a negatively charged side chain and include glutamic acid (E) and aspartic acid (D), and 3) “positively charged polar amino acids” which have a positively charged side chain and include lysine (K), arginine (R), and histidine (H).

The term “nucleic acid molecule” means a polymer of any length of deoxyribonucleic acid (DNA), or polydeoxyribonucleotides, notably including complementary DNAs or cDNAs, genomic DNAs, plasmids, vectors, viral genomes, isolated DNA, probes, primers and any mixture thereof; or a polymer of any length of ribonucleic acid (RNA), or polyribonucleotides, notably including messenger RNA or mRNA, antisense RNA; or mixed polyribo-polydeoxyribonucleotides. They include single- or double-stranded, linear or circular, natural or synthetic polynucleotides. In addition, a polynucleotide may comprise non-natural nucleotides and may be interrupted with non-nucleotide components.

In the context of the present invention, the terms “nucleic acid”, “nucleic acid molecule”, “polynucleotide” and “nucleotide sequence” are used interchangeably.

The term “isolated molecule” means a molecule, notably a protein, polypeptide, peptide, nucleic acid molecule, plasmid vector, viral vector or host cell, which is extracted from its natural environment (i.e. separated from at least one other component with which it is naturally associated).

The terms “polypeptide”, “protein” and “peptide” mean polymers of amino acid residues which comprise at least nine amino acids bound by peptide bonds. The polymer may be linear, branched or cyclic. The polymer may comprise natural amino acids and/or amino acid analogues and may be interrupted with non-amino acid residues. As a general indication and without however being bound thereto in the present patent application, if the amino acid polymer contains more than 50 amino acid residues, it is preferably called a polypeptide or protein, whereas if the polymer consists of 50 or fewer amino acids, it is preferably called a “peptide”.

The term “identity” means an exact sequence match between two polypeptides or two amino acid molecules. The “percentages of identity” referred to in the context of the description of the present invention are determined on the basis of a global alignment of the sequences (nucleic or protein) to be compared, i.e. an alignment of the sequences taken in their entirety over their entire length, using any algorithm well known to those skilled in the art, such as the Needleman-Wunsch algorithm (1970). This sequence comparison may be performed using any software program well known to those skilled in the art, for example the needle software program, using the “Gap open” parameter equal to 10.0, the “Gap extend” parameter equal to 0.5 and a “Blosum 62” matrix. The needle software is, for example, available on the ebi.ac.uk web site under the name “Align”.

The term “vector” means a vehicle, preferably a nucleic acid molecule or a viral particle, which contains the elements required to allow the administration, propagation and/or expression of one or more nucleic acid molecules in a host cell or organism.

Functionally, this term encompasses vectors for maintenance (cloning vectors), vectors for expression in various host cells or organisms (expression vectors), extrachromosomal vectors (for example multicopy plasmids) or integration vectors (for example designed to integrate into the genome of a host cell and produce additional copies of the nucleic acid molecule it contains when the host cell replicates). The term also encompasses shuttle vectors (for example, operating in both prokaryotic and/or eukaryotic hosts) and transfer vectors (for example, for transferring nucleic acid molecule(s) into the genome of a host cell).

Structurally, the vectors according to the invention may be natural, synthetic or artificial genetic sources, or a combination of natural and artificial genetic elements.

Thus, in the context of the invention, the term “vector” should be understood broadly as including both plasmid and viral vectors.

A “plasmid” as used herein denotes a replicable DNA construct. Usually, plasmid vectors contain selection marker genes which allow host cells bearing the plasmid to be positively or negatively identified and/or selected in the presence of the compound corresponding to the selection marker. A variety of positive and negative selection marker genes are known in the art. For illustrative purposes, an antibiotic resistance gene may be used as a positive selection marker gene allowing a host cell to be selected in the presence of the corresponding antibiotic.

The term “viral vector” as used herein refers to a nucleic acid vector which comprises at least one element of a virus genome and can be packaged into a viral particle or a viral particle. Viral vectors may be replication-competent or selective (for example, designed to replicate better or selectively in specific host cells), or may be genetically inactivated so as to be defective or deficient for replication.

The term “host cell” means a cell containing a heterologous nucleic acid molecule. The term “heterologous” or “exogenous” means that the nucleic acid molecule comes from a different species to that of the host cell. Thus, a host cell is not a naturally occurring cell, but a molecular biology tool obtained via genetic manipulation techniques.

The host cell may consist of a single cell type, or a group of different cell types (in which case there is a mixture of different host cell types). The host cell may also be a hybrid cell, i.e. resulting from the fusion of at least two cells of different types. The host cell may belong to cultured cell lines, primary cells, stem cells or proliferative cells. In the context of the invention, the term “host cell” comprises both prokaryotic and eukaryotic cells. The term “prokaryote” means a unicellular microorganism whose cell structure does not include a nucleus. Prokaryotes include the bacterial and archaeal kingdoms. The term “bacterium” means a microscopic prokaryotic organism present in all media. The term “eukaryote”, in contrast to prokaryotes, means any unicellular or multicellular organism whose cells have a structured nucleus. Eukaryotic cells include yeast, insect, plant and animal cells (notably non-human mammalian cells). The host cell may, for example, be isolated or organized as a tissue, organ or even as part of a complete organism. In the case where the host cell is within a complete organism, said organism is not human.

In the detailed description which follows, the embodiments may be taken alone or in any appropriate combination by a person skilled in the art.

### Use of Isolated Polypeptides, Nucleic Acid Molecules, Vectors or Host Cells for the Production of Phloroglucinol

The focus herein is on the use of isolated polypeptides chosen from type III polyketide synthases from ascomycete fungi.

Specifically, in an entirely surprising manner, the Inventors have identified genes encoding new type III polyketide synthases in the genome of ascomycete fungi. Furthermore, type III polyketide synthases are a broad class of enzymes collating proteins with sequence similarities while at the same time having very dissimilar enzymatic activities (Meslet-Cladière et al., 2013). Thus, phloroglucinol synthase activity is just one of the multiple activities which may be present in a type III polyketide synthase. In addition, no sequence unit associated with phloroglucinol synthase activity has been described, making the identification of type III polyketide synthases having such activity particularly difficult. However, the Inventors have also demonstrated that the new type III polyketide synthases identified in ascomycete fungi have phloroglucinol synthase activity.

In a first aspect, the invention thus relates to the use of a polypeptide chosen from type III polyketide synthases from ascomycete fungi, to an isolated nucleic acid molecule encoding same, to a vector comprising a nucleic acid molecule encoding same or to a non-human host cell expressing same, for producing phloroglucinol.

### Polypeptide Used

The polypeptide used in the invention is chosen from type III polyketide synthases from ascomycete fungi, and advantageously has phloroglucinol synthase activity.

It may notably be chosen from type III polyketide synthases from fungi of the families Ophiocordycipitaceae and Trichocomaceae, advantageously from the genera Hirsutella and Aspergillus, more advantageously from the species Hirsutella minnesotensis 3608 and Aspergillus tanneri.

Advantageously, the polypeptide used in the invention corresponds to those identified herein as having phloroglucinol synthase activity or to derivatives thereof. In particular, the polypeptide used in the invention comprises, consists essentially of or is constituted of an amino acid sequence having at least 70% identity, at least 75% identity, advantageously at least 80% identity, at least 85% identity, more advantageously at least 90% identity, at least 91% identity, at least 92% identity, at least 93% identity, at least 94% identity, even more advantageously at least 95% identity, at least 96% identity, at least 97% identity, at least 98% identity, at least 99% identity, at least 99.5% identity, or even 100% identity with a sequence chosen from SEQ ID NO: 1 (PhlD de Hirsutella minnesotensis 3608 designated PlhD-Hm2), SEQ ID NO: 2 (PhlD from Hirsutella minnesotensis 3608 designated PlhD-Hm1) and SEQ ID NO: 3 (PhlD from Aspergillus tanneri designated PlhD-At).

The sequences SEQ ID NO: 1 to 3 are shown in Table 1 below:

TABLE 1

| | | Species | |
|---|---|---|---|
| Identifier | Name | of origin | Amino acid sequence |
| SEQ ID | PHLD- | Hirsutella | MAPMLHVPHTADIGETTRGGSNNVTSEPRNANRITSRQVCPVRTLVIEGIATGVP |
| NO: 1 | Hm2 | minnesotensis | QESTRQKDAAEKVCALFQAEPAQQERINRLYTKTQIDKRHMTVDPLQKKFDRSM |
| | | 3608 | SIRQRMDLFLQHASPLAIDVSSRALDKAQATAEDIGLLVLVTSTGFIAPGVDVTVM |
| | | | KALGLSPSVSRVTVNFMGCAAAMNGLRAATDFVRAHPRSKALMCCVELSSVNAV |
| | | | FANNINDVIISSLFADGCAAMVVGSRDNQEGQDPLRPGQVIVHEHFCQFVEGSED |
| | | | GITLGVNDNGITCELSAKLPKYIRSGLGPVVCSLLDRHGMTKSDVNYWAIHPGGP |
| | | | KIIEESVCSLDIPAHVATKSWDVLAEYGNMLSASLPFVLQRMVDEANSEGSSGKGV |
| | | | AFSFAPGVTVEGILFEIPECT |
| SEQ ID | PHLD- | Hirsutella | MAAQSGPGPLVVIDSIATGVPEITRKQAESANRVAKLMKVDQEQHKRISCIYAKTR |
| NO: 2 | Hm1 | minnesotensis | IHTRHMAVDPLSADFDRSLTIRERMNRFLEYAAPLATDVSGRALAEAQVQPAEVG |
| | | 3608 | MLILVTSTGFIAPGVDVAVMKGLGLSPSAPRAVVNFMGCAAAMNGLRLASNFVR |
| | | | GYPGAKALVCCVELSSVNAVWANNINDTIISSLFADGCAAMVVGSTTSAKLVPGK |
| | | | VVIREQFNQFVENSVDGITLGVNCDGITCDLSAKLPLYIRGGLDPVISKVLARHGM |
| | | | AKSDIDLWAIHPGGPKIIEESIRSLGVDEQVATKSWEVLAEHGNMLSASLPFVLKR |
| | | | MLAEARLDKPVSSGVAFSFAPGVTVEGFLFDVVTEEG |
| SEQ ID | PHLD- | Aspergillus | MASSSNINTSTCVPLYPKDEVSKFHGRVPNNMKDLYGEDAPQAQADIRGIGFASP |
| NO: 3 | At | tanneri | QHQQLGNKPLIVIESIATEVPEGKRTQSDTAHVVASLFDSHPEQRDRIPRLYAKTRI |
| | | | HTRHMAVDPLHPKFDRAMPIRKRMDLFLEHAAPLAIKVSSRALTASRVINPAEDIG |
| | | | MLVLVTSTGFVAPGVDVAIVKGLGLSHSVSRAVVNFMGCAAAMNGLRLAADFVR |
| | | | AHPDSKALVCCVELSSVNAVYNDDLNDVIISSLFADGSAAMVVGSPSPTCPGHGQ |
| | | | QKMHLMPGEVIIRQNFHNLVDETSDGITLGINHNGITCELSPDLPSYIYAGLAPVID |
| | | | KVLALHEMSRSDVHLWAIHPGGPKIIEESVRSLGLNNQNAAKSWEVLMKFGNMLS |
| | | | ASLPFVLEQMVAEAKDERPLSRGLAFSFAPGITVEGIVFEVVGTGIDQSVSIC |

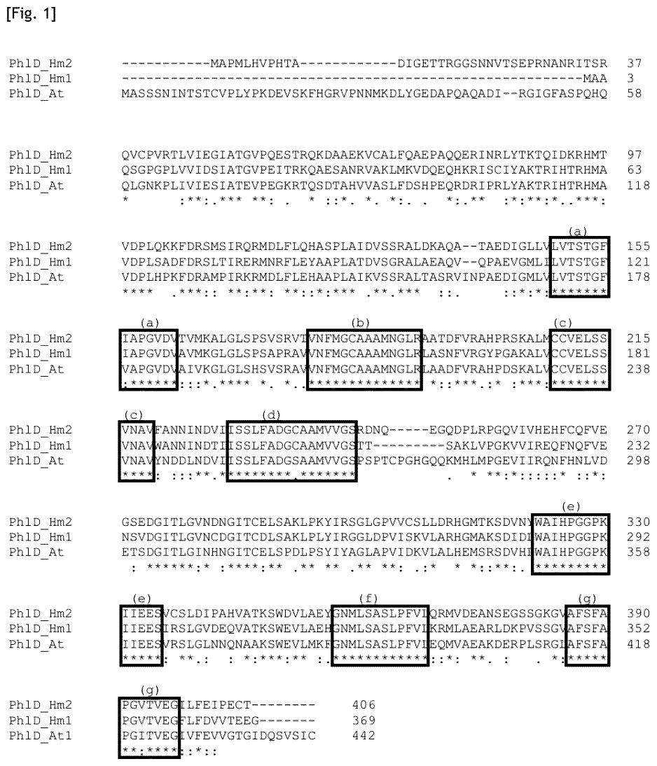

In addition, the Inventors have identified units conserved between the three sequences SEQ ID NO: 1, SEQ ID NO: 2 and SEQ ID NO: 3 (see the alignment in FIG. 1), which are therefore advantageously present in the polypeptide sequence used in the invention.

Thus, in an advantageous general embodiment, the polypeptide used in the invention also comprises (in addition to a minimum percentage of identity with SEQ ID NO: 1, SEQ ID NO: 2 or SEQ ID NO: 3 as described above) at least one fragment chosen from:
(a) LVTSTGFX₁APGVDV, in which X₁ is chosen from apolar amino acids (i.e. glycine (G), alanine (A), valine (V), leucine (L), isoleucine (I), proline (P), tryptophan (W), phenylalanine (F), tyrosine (Y), methionine (M) and cysteine (C), SEQ ID NO: 4);

(b)
(SEQ ID NO: 5)
VNFMGCAAAMNGLR;
(c)
(SEQ ID NO: 6)
CCVELSSVNAV;

(d) ISSLFADGX₂AAMVVGS, in which X₂ is chosen from apolar or neutral polar amino acids (i.e. glycine (G), alanine (A), valine (V), leucine (L), isoleucine (I), proline (P), tryptophan (W), phenylalanine (F), tyrosine (Y), methionine (M), cysteine (C), serine (S), threonine (T), asparagine (N), and glutamine (Q)), (SEQ ID NO: 7);

(e)
(SEQ ID NO: 8)
WAIHPGGPKIIEES;
(f)
(SEQ ID NO: 9)
GNMLSASLPFVL;
and

(g) AFSFAPGX₃TVEG, in which X₃ is chosen from apolar amino acids (i.e. glycine (G), alanine (A), valine (V), leucine (L), isoleucine (I), proline (P), tryptophan (W), phenylalanine (F), tyrosine (Y), methionine (M) and cysteine (C)), SEQ ID NO: 10).

Advantageously, in an intermediate embodiment, the polypeptide used in the invention also comprises (in addition to a minimum percentage of identity with SEQ ID NO: 1, SEQ ID NO: 2 or SEQ ID NO: 3 as described above) at least one fragment chosen from:
(a) LVTSTGFX₁APGVDV, in which X₁ is chosen from aliphatic apolar amino acids (i.e. alanine (A), valine (V), leucine (L), isoleucine (I), and proline (P), SEQ ID NO: 11);

(b)
(SEQ ID NO: 5)
VNFMGCAAAMNGLR;
(c)
(SEQ ID NO: 6)
CCVELSSVNAV;

(d) ISSLFADGX₂AAMVVGS, in which X₂ is chosen from sulfur-based apolar or neutral polar amino acids (i.e. methionine (M), cysteine (C), serine (S), threonine (T), asparagine (N) and glutamine (Q)), SEQ ID NO: 12);

(e)
(SEQ ID NO: 8)
WAIHPGGPKIIEES;
(f)
(SEQ ID NO: 9)
GNMLSASLPFVL;
and

(g) AFSFAPGX₃TVEG, in which X₃ is chosen from aliphatic apolar amino acids (i.e. alanine (A), valine (V), leucine (L), isoleucine (I), and proline (P)), SEQ ID NO: 13).

Even more advantageously, in a limited embodiment, the polypeptide used in the invention also comprises (in addition to a minimum percentage of identity with SEQ ID NO: 1, SEQ ID NO: 2 or SEQ ID NO: 3 as described above) at least one fragment chosen from:
(a) LVTSTGFX₁APGVDV, in which X₁ is chosen from valine and isoleucine (SEQ ID NO: 14);

(b)
(SEQ ID NO: 5)
VNFMGCAAAMNGLR;
(c)
(SEQ ID NO: 6)
CCVELSSVNAV;

(d) ISSLFADGX₂AAMVVGS, in which X₂ is chosen from cysteine and serine (SEQ ID NO: 15);

(e)
(SEQ ID NO: 8)
WAIHPGGPKIIEES;
(f)
(SEQ ID NO: 9)
GNMLSASLPFVL;
and

(g) AFSFAPGX₃TVEG, in which X₃ is chosen from valine and isoleucine (SEQ ID NO: 16).

Particularly advantageously, in a very limited embodiment, the polypeptide used in the invention also comprises (in addition to a minimum percentage of identity with SEQ ID NO: 1, SEQ ID NO: 2 or SEQ ID NO: 3 as described above) at least one fragment chosen from:

(a)
(SEQ ID NO: 17)
LVTSTGFIAPGVDV;
(b)
(SEQ ID NO: 5)
VNFMGCAAAMNGLR;
(c)
(SEQ ID NO: 6)
CCVELSSVNAV;
(d)
(SEQ ID NO: 18)
ISSLFADGCAAMVVGS;
(e)
(SEQ ID NO: 8)
WAIHPGGPKIIEES;
(f)
(SEQ ID NO: 9)
GNMLSASLPFVL;
and
(g)
(SEQ ID NO : 19)
AFSFAPGVTVEG.

Advantageously, the polypeptide used in the invention also comprises (in addition to a minimum percentage of identity with SEQ ID NO: 1, SEQ ID NO: 2 or SEQ ID NO: 3 as described above) several of the fragments (a) to (g) as described above (general, intermediate, limited or very limited embodiment). In particular, the polypeptide used in the invention may also comprise (in addition to a minimum percentage of identity with SEQ ID NO: 1, SEQ ID NO: 2 or SEQ ID NO: 3 as described above (general, intermediate or limited embodiment)):
fragments (a), (b) and (d), or
all the fragments (a) to (g).

In a particularly advantageous manner, the polypeptide used in the invention comprises, consists essentially of or is constituted of an amino acid sequence chosen from SEQ ID NO: 1, SEQ ID NO: 2 and SEQ ID NO: 3; preferably, the polypeptide used in the invention comprises, consists essentially of or is constituted of the amino acid sequence SEQ ID NO: 1.

### Isolated Nucleic Acid Molecule Used

The isolated nucleic acid molecule used in the invention may be any nucleic acid molecule comprising a nucleic acid sequence encoding any polypeptide chosen from the type III polyketide synthases from ascomycete fungi as defined above.

According to one embodiment, the isolated nucleic acid molecule also comprises a promoter controlling the expression of the nucleic acid sequence encoding the polypeptide chosen from the type III polyketide synthases from ascomycete fungi as defined above. Advantageously, the promoter is an exogenous promoter, in particular a yeast promoter, preferably a promoter chosen from ADH2 (pADH2, this promoter allows expression notably when the culture medium contains ethanol as carbon source), CCW12 (pCCW12, this promoter allows expression notably when the culture medium contains glucose as carbon source), and TEF1 (pTEF1, this promoter allows expression notably when the culture medium contains glucose or sucrose as carbon source), more preferably a promoter chosen from ADH2 (pADH2) from Saccharomyces cerevisiae, CCW12 (pCCW12) from S. cerevisiae, and TEF1 (pTEF1) from S. cerevisiae, even more preferably a promoter chosen from ADH2 of sequence SEQ ID NO: 20, CCW12 of sequence SEQ ID NO: 21 and TEF1 of sequence SEQ ID NO: 22.

According to an embodiment which is alternative to or combinable with the preceding one, the isolated nucleic acid molecule also comprises a transcription terminator for the nucleic acid sequence encoding the polypeptide chosen from the type III polyketide synthases from ascomycete fungi as defined above. Advantageously, the terminator is an exogenous terminator, in particular a yeast terminator, preferably the RPL3 terminator (tRPL3) or the ADH1 terminator, more preferably the RPL3 terminator of S. cerevisiae or the ADH1 terminator of S. cerevisiae, more preferably the RPL3 terminator of sequence SEQ ID NO: 23 or the ADH1 terminator of sequence SEQ ID NO: 24.

According to a preferred embodiment, the isolated nucleic acid molecule also comprises both a promoter and a terminator which are as defined above.

According to an embodiment which is alternative to or combinable with each of the embodiments directed towards the isolated nucleic acid molecule used in the invention, the nucleic acid molecule also comprises an export sequence. Advantageously, this export sequence allows the secretion or excretion of the polypeptide(s) encoded by the nucleic acid molecule into the cellular medium.

According to a preferred embodiment, the isolated nucleic acid molecule also comprises a promoter, terminator and also an export sequence as defined above.

The nucleic acid molecule may be isolated from homologous strains in culture, preferably chosen from the genera Hirsutella and Aspergillus, more advantageously from the species Hirsutella minnesotensis 3608 and Aspergillus tanneri. Alternatively, the nucleic acid molecule may be isolated from a heterologous vector or host cell comprising said molecule, said vector or host cell being as defined above and as described hereinbelow in the sections “Host cells” or “Vectors”. Alternatively, the isolated nucleic acid molecule may be synthesized in vitro via nucleic acid synthesis techniques known to those skilled in the art.

According to an embodiment which is alternative to or combinable with each of the embodiments directed towards the isolated nucleic acid molecule used in the invention, the nucleic acid sequence included in the isolated nucleic acid molecule used in the invention is also optimized for expression in a host cell, in particular in a yeast or in a bacterium.

To optimize expression in a host cell, in particular in a yeast or a bacterium, a person skilled in the art knows how to use the genetic code's usual bias, which denotes the preferential use, for a given organism, of one of the possible triplets of nucleotides or codons coding for the same amino acid. Specifically, there are generally several combinations of three nucleotides (called “codons”) encoding the same amino acid (except for methionine and tryptophan), known as synonymous codons, but some of these combinations are in general preferentially used by a given organism. For the production of an amino acid sequence of interest, optimal expression can thus be obtained when the codons chosen to encode the amino acid sequence are those preferentially used by the host cell's organism of origin. Depending on the chosen production organism (notably a yeast or a bacterium), different optimal nucleic acid sequences will thus be used when the nucleic acid sequence included in the isolated nucleic acid molecule used in the invention is also optimized for expression in the chosen production organism.

Various software packages are accessible to a skilled person to optimize codons for expression in a host cell, notably a yeast or in a bacterium. Examples of such software packages include Twist Codon Optimization tool software (supplied by Twist Biosciences), the GenSmart™ Codon Optimization software (supplied by GenScript and whose functionality is described in patent application WO2020/024917A1), the IDT Codon Optimization Tool software (supplied by Integrated DNA Technologies), and Azenta's codon optimization tool software (supplied by Azenta).

Examples of optimized nucleic acid sequences respectively encoding the polypeptides having the amino acid sequence SEQ ID NO: 1 to 3 are the nucleic acid sequences SEQ ID NO: 25 to SEQ ID NO: 27, respectively, shown in Table 2 below and which are optimized (at least partially) for expression in a yeast, notably Saccharomyces cerevisiae:

TABLE 2

| | Amino acid | |
|---|---|---|
| | sequence | Nucleic acid sequence optimized for expression in a yeast, notably |
| Name | identifier | Saccharomyces cerevisiae (identifier) |
| PHLD- | SEQ ID NO: 1 | atggctccaatgctacatgttcctcataccgcggatattggtgaaacaactaggggcggctcgaataa |
| Hm2 | | cgtcacaagtgagcctaggaacgcgaataggatcaccagcagacaagtttgcccagtaagaactctt |
| | | gttattgaggggatcgccaccggtgtaccccaagaatctaccaggcagaaagacgcagctgaaaaa |
| | | gtttgtgccctatttcaagcagaaccggcacaacaggaaagaatcaatagattatacaccaaaaccc |
| | | aaatagacaagagacacatgacagtagatcctttacaaaaaaagtttgatagaagtatgagcatcag |
| | | gcagagaatggatcttttcttacaacacgctagtccgttagcaattgacgtgtcttccagggctttaga |
| | | taaggcgcaagcaacagctgaggatattggtcttcttgttttggtaacatcgactggctttatagctcc |
| | | aggagtggatgtaactgtcatgaaagctttaggattgtcgccaagtgtttcaagggtaacggtaaact |
| | | tcatgggttgcgcagccgcgatgaacggtctaagagctgccacagattttgtgagagcacaccctcgt |
| | | tcaaaagcattaatgtgctgtgtcgaattgagttctgtaaatgcagtttttgccaacaacattaacgat |
| | | gttattatttcgtccttgttcgcagacggctgtgcagctatggtcgttggatccagagacaatcaagaa |
| | | ggtcaagaccctttgaggcctgggcaggtgattgtgcatgaacatttttgtcagtttgtagaaggctca |
| | | gaggatgggataactttgggagtgaacgacaatggaatcacctgtgaattgagtgctaaactaccaa |
| | | agtatataaggtcgggtctaggtccagttgtatgctctttattggatagacatggcatgacaaaatcag |
| | | acgttaattattgggcaatccatccaggtggacctaagataatagaggaatccgtctgctccctagac |
| | | attccagcccacgtggcgactaaatcttgggatgtactggctgaatatggtaacatgttatctgcatct |
| | | ctacccttcgtacttcagaggatggttgatgaagcaaatagtgaaggttcatcaggcaaaggggttgc |
| | | tttttcgtttgcccctggtgtaaccgttgaaggcattctattcgaaataccagaatgcacttaa (SEQ |
| | | ID NO: 25) |
| PHLD- | SEQ ID NO: 2 | atggctgcccaatcaggtccaggtccattggttgtaattgatagtatcgctaccggtgtacccgaaat |
| Hm1 | | aacgagaaaacaagctgaaagcgccaacagggtggctaaattaatgaaagttgatcaagagcaaca |
| | | taagcgtatttcctgtatctatgcaaaaactaggattcacactaggcacatggctgtcgatccattaa |
| | | gcgcagactttgatcgttcccttacaatcagggaaagaatgaatagatttttagaatatgcagcccca |
| | | ttagcaacagatgtttcgggtagggctctagccgaggcacaggtccaaccagctgaagttggaatgtt |
| | | aattttggtaacatctactggatttattgcacccggtgtcgatgttgcagtaatgaaaggtttgggcct |
| | | gtctccaagcgccccaagagcagttgtaaattttatggggtgcgctgctgctatgaatggtctaaggtt |
| | | ggcttcaaattttgtccgtggctacccaggcgccaaggcattagtctgctgtgtggagctgtcatctgt |
| | | gaacgctgtgtgggctaacaacattaacgatacgataatctcttcattgtttgcggacggctgcgccg |
| | | ctatggttgttggctcaaccaccagtgccaaacttgttcctggtaaagtggttattagggaacaattca |
| | | atcaatttgtcgaaaattccgtcgatgggattacattaggggtcaattgtgatggaattacatgcgact |
| | | tgtctgccaaactacctttatatatccgtggcggcttagatccagtaatatcgaaagttctagctaggc |
| | | atggaatggctaagagtgatatcgatctgtgggccatacatccaggtggtcctaagatcattgaagaa |
| | | tcgatacgttccttgggagttgatgaacaggttgcaactaaatcttgggaagtactggcagaacacgg |
| | | taacatgctttcggcatccctgccatttgtcctgaaacgtatgcttgcagaagctagattagataagcc |
| | | cgtttcctctggggtggccttttctttcgcaccgggtgtaaccgttgaaggatttttgttcgatgtcgtaa |
| | | ccgaagagggatga (SEQ ID NO: 26) |
| PHLD- | SEQ ID NO: 3 | atggcttccagttccaatattaatacttctacttgtgttccattatacccaaaggacgaagtatcaaag |
| At | | tttcacggaagggtgccaaataacatgaaagatttgtatggcgaagatgcaccacaggctcaggccg |
| | | atatcagaggcattggctttgcgtcgccacagcatcaacaactgggcaataagcctttaatagttata |
| | | gaatccatcgctaccgaggttccggaaggaaaacgtactcaaagtgatacagcacatgtggtcgcct |
| | | cgctttttgattcccatccagagcagagggatcgtatcccacgtttatacgccaaaaccaggattcat |
| | | acgagacatatggccgttgacccattgcatccaaagttcgatagggccatgccaatccgtaaacgtat |
| | | ggatttgttcttagaacatgctgcacctttagccatcaaggttagctccagagctttgactgcttctag |
| | | ggttattaaccccgcggaagatataggtatgttggttttagtgaccagtacgggctttgttgctccggg |
| | | agttgatgttgctattgtcaagggtcttggcttgtcccattctgttagccgtgctgtagtgaattttatgg |
| | | gttgtgcagcagctatgaacggactaagattggctgccgactttgtcagggcccatcctgacagcaag |
| | | gctctagtttgctgcgttgagctgagttccgtgaatgcggtgtacaatgatgatttaaacgacgtcatt |
| | | atttcatcattgtttgctgacggctcagcagctatggtcgtaggatctccatcgcccacgtgtcccggt |
| | | catggtcagcaaaagatgcacttgatgcctggcgaagttataattcgtcagaatttccacaatttggt |
| | | agatgaaaccagtgacggaataacactggggattaaccacaatggtattacctgtgagttaagtcca |
| | | gatttaccttcatacatttacgcaggcctagcgcccgttatagacaaagtcctggcattacacgaaat |
| | | gtcccgtagcgatgtacatttgtgggctattcaccctggtggtcctaagattatcgaagagtcagttag |
| | | atccttgggacttaataatcaaaatgctgctaaatcctgggaagtcttgatgaaattcggcaatatgct |
| | | gagtgcaagtctgccatttgtattagaacagatggtggcagaagctaaagacgagagaccgctatct |
| | | aggggattggctttttccttcgctcctgggattactgttgaaggaatcgtgtttgaagttgtcggaacgg |
| | | gaatcgatcaatcagtttcgatttgttag (SEQ ID NO: 27) |

Nevertheless, a person skilled in the art will know how to generate other nucleic acid sequences optimized for expression in a yeast, notably Saccharomyces cerevisiae, or in another type of host cell (notably another yeast species or a bacterium).

Once the optimized nucleic acid sequence has been defined, a person skilled in the art can obtain this sequence by in vitro synthesis directly with the optimized codons. When the number of nucleotides to be modified relative to the original sequence is not too high, the optimized sequence can also be obtained by in vitro site-directed mutagenesis of a sample of the nucleic acid molecule whose codons are to be adapted, using polymerase chain reaction (PCR) amplification.

According to a preferred embodiment, the isolated nucleic acid molecule also comprises both a promoter and a terminator as defined above, and the nucleic acid sequence included in the isolated nucleic acid molecule used in the invention is also optimized for expression in a host cell, advantageously in a yeast or a bacterium, preferably in a yeast, as described above.

According to another preferred embodiment, the isolated nucleic acid molecule also comprises a promoter, a terminator and also an export sequence as defined above, and the nucleic acid sequence included in the isolated nucleic acid molecule used in the invention is also optimized for expression in a host cell, advantageously in a yeast or a bacterium, preferably in a yeast, as described above.

### Vector Used

The vector used in the invention may be any vector comprising any nucleic acid molecule as defined above.

Vectors that are suitable in the context of the present invention include, without limitation, bacteriophage vectors, plasmids or cosmids for expression in prokaryotic host cells such as bacteria (for example E. coli, or bacteria of the genus Pseudomonas); vectors for expression in yeast (for example Saccharomyces cerevisiae, Schyzosaccharomyces pombe, Pichia pastoris); baculovirus vectors for expression in insect cell systems (for example, Sf 9 cells); viral and plasmid vectors for expression in plant cell systems (for example, Ti plasmid, Cauliflower mosaic virus, CaMV, Tobacco mosaic virus TMV); and also viral and plasmid vectors for expression in vertebrate cells or organisms, notably mammals.

Depending on the host of interest, a person skilled in the art is familiar with suitable expression vectors. These vectors are generally commercially available (for example from suppliers such as Invitrogen, Stratagene, Amersham Biosciences, Promega, etc.), available from deposition institutions such as the American Type Culture Collection (ATCC, Rockville, MD), or have been the subject of numerous publications describing their sequence, structures and production methods, so that a person skilled in the art can apply them without difficulty.

The invention most particularly relates to the production of phloroglucinol in yeasts, and thus the vector used in the invention is advantageously suitable and even optimized for yeast transfection. The vector used in the invention may notably advantageously be a plasmid vector suitable for yeast transfection. Representative examples of plasmid vectors suitable for yeast transfection include, without limitation, pREP4, pCEP4 (Invitrogen), pCI (Promega), pVAX (Invitrogen), pgWiz (Gene Therapy System Inc.) and YCplac22 (ATCC 87585), and also any derivative of these vectors (notably a derivative of YCplac22 into which the pair (PGK promoter and CYC terminators) and also the pair (TEF1 promoter and ADH1 terminator) have been integrated), but any other vector suitable for yeast transfection may be used.

### Host Cell Used

The host cell used in the invention may be any host cell expressing any polypeptide chosen from the type III polyketide synthases from ascomycete fungi as defined above. It may notably be chosen from host cells comprising an isolated nucleic acid molecule as defined above or a vector as defined above.

As defined above, a host cell is a cell containing a heterologous nucleic acid molecule. In the context of the invention, the heterologous nucleic acid molecule corresponds to the isolated nucleic acid molecule as defined above, or to the vector as defined above.

The host cell used may be a prokaryotic cell or a eukaryotic cell. Among prokaryotic cells, it may be chosen from bacteria. Among eukaryotic cells, it may be chosen from yeast cells, fungal cells, algal cells, insect cells, plant cells or non-human mammalian cells.

The host cell used is preferably a yeast, said yeast being particularly selected from the genera Saccharomyces, Candida, Eremothecium, Dekkera, Pichia (Hansenula), Debaryomyces, Lodderomyces, Yarrowia, Zigosaccharomyces, Schizosaccharomyces, Torulaspora, Kluyveromyces, Brettanomycces, Cryptococcus and Malassezia.

Even more particularly, the yeast is selected from the species Saccharomyces cerevisiae, Saccharomyces boulardii, Saccharomyces douglasii, Saccharomyces bayanus, Zigosaccharomyces bailii, Schizosaccharomyces pombe, Dekkera brucelensis, Dekkera intermedia, Brettanomycces custersii, Brettanomycces intermedius, Kluyveromyces themotolerens, Torulaspora globosa and Torulaspora glabrata.

Even more particularly, the yeast is of the Saccharomyces genus, preferably of the Saccharomyces cerevisiae species.

The host cell used may also be a bacterium, said bacterium being particularly selected from proteobacteria, Actinomycetes bacteria and Firmicutes bacteria. Among proteobacteria, the host cell may advantageously be chosen from the genera Escherichia (notably from strains of the species Escherichia coli, the most widely used species for recombinant protein production) and Pseudomonas. Among Actinomycetes bacteria, the host cell may advantageously be chosen from the Streptomyces and Corynebacterium genera. Among Firmicutes bacteria, the host cell may advantageously be chosen from the Bacillus and Lactobacillus genera.

The host cell used comprises at least one copy of the isolated nucleic acid molecule as defined above integrated into its genome. It may notably comprise a single copy of the isolated nucleic acid molecule as defined above integrated into its genome.

When the host cell used is a yeast cell (notably of the genus Saccharomyces, preferably of the species Saccharomyces cerevisiae), the copy or copies of the nucleic acid molecule may be integrated into different loci, preferentially at the URA3 locus, the JLP1 locus, the LEU2 locus, or the TRP1 locus of the genome of said yeast cell. When the host cell is a yeast cell and when several copies of the nucleic acid molecule are integrated, the different copies may be integrated into the same locus, or even into different loci, preferentially into any combination of the URA3, JLP1, LEU2, and/or TRP1 loci.

Advantageously, the codons used in the nucleic acid molecule encoding the polypeptide chosen from the type III polyketide synthases from ascomycete fungi included in the host cell or in the vector included in the host cell have been adapted for optimal expression in the selected host cell. As previously explained, for the production of an amino acid sequence of interest, optimal expression can be obtained when the codons chosen to encode the amino acid sequence are those preferentially used by the host cell organism of origin. Irrespective of the type of host cell, a person skilled in the art will know which codons are to be favoured from the literature or by using codon optimization software. The examples of codon optimization software mentioned above for yeast may also be used for other host cell types. Yeasts, and notably those of the Saccharomyces genus, preferably of the Saccharomyces cerevisiae species, being most particularly preferred as host cells, the codons used in the nucleic acid molecule encoding the polypeptide chosen from the type III polyketide synthases from ascomycete fungi included in the host cell or in the vector included in the host cell have advantageously been adapted for optimal expression in yeasts, and notably in yeasts of the genus Saccharomyces, preferably of the species Saccharomyces cerevisiae.

For the use according to the invention, the host cells can be cultured in aerobic or anaerobic bioreactors, on a small or large scale, in bottles or Petri dishes. Cultivation may be performed at a temperature and a pH, in a culture medium and at an oxygen content suitable for a given host cell. For yeasts, and more particularly for Saccharomyces cerevisiae, cultivation is advantageously performed via any suitable method. Processes for culturing a Saccharomyces cerevisiae strain are known in the art, and a person skilled in the art knows how to optimize the culture conditions for each strain as a function of its nature. Conventional processes are notably described in the reference work “Yeast Technology”, 2nd Edition, 1991, Reed and Nagodawithana, published by Van Nostrand Reinhold (ISBN 0-442-31892-8). In particular, the culturing of a yeast strain, in particular of the Saccharomyces cerevisiae species, may generally be performed at a temperature of between 2° and 37° C., in a rich liquid medium (for example, the YPD medium available from VWR) or a synthetic medium (defined to precisely meet the needs of the strain), in aerobic or anaerobic culture.

### Use

The polypeptide chosen from type III polyketide synthases from ascomycete fungi, the nucleic acid molecule encoding same, the vector comprising a nucleic acid molecule encoding same or the host cell expressing same, each as defined above, is used to produce phloroglucinol, irrespective of the production method used.

Depending on the method, a person skilled in the art will know how to choose the most appropriate between the polypeptide chosen from type III polyketide synthases from ascomycete fungi, the nucleic acid molecule encoding same, the vector comprising a nucleic acid molecule encoding same or the host cell expressing same, each as defined above.

In particular, the polypeptide chosen from the type III polyketide synthases from ascomycete fungi as described above can be used directly to produce phloroglucinol in vitro, provided that it is brought into contact with malonyl-CoA. Specifically, phloroglucinol synthases perform the condensation of three malonyl-CoA molecules to form one phloroglucinol molecule according to the Reaction I reaction scheme described previously.

The nucleic acid molecule encoding same and the vector comprising a nucleic acid molecule encoding same may be used to transfect a host cell, which may then be used either to produce the polypeptide (which can then produce phloroglucinol in vitro) or to directly produce phloroglucinol when it is cultured in the presence of a suitable substrate (such as a carbon source like glucose or ethanol).

Examples of more precise methods in which the polypeptide chosen from type III polyketide synthases from ascomycete fungi, the nucleic acid molecule encoding same, the vector comprising a nucleic acid molecule encoding same or the host cell expressing same as described above are described in the section below concerning methods according to the invention for producing phloroglucinol.

### Isolated Nucleic Acid Molecule According to the Invention

The invention also relates to an isolated nucleic acid molecule comprising a nucleic acid sequence encoding a polypeptide chosen from the type III polyketide synthases from ascomycete fungi as defined above, characterized in that:
a) the isolated nucleic acid molecule also comprises a promoter controlling the expression of the nucleic acid sequence; or
b) the isolated nucleic acid molecule also comprises a transcription terminator controlling the expression of the nucleic acid sequence; or
c) the nucleic acid sequence is also optimized for expression in a host cell, notably in a yeast or in a bacterium; or
d) any combination of a) to c).

Such isolated nucleic acid molecules are useful either for the production of a polypeptide chosen from the type III polyketide synthases from ascomycete fungi as defined above, or for the direct production of phloroglucinol when they are transfected (directly or within a vector) into a host cell, grown in the presence of a suitable substrate.

In embodiment a), the isolated nucleic acid molecule also comprises a promoter controlling the expression of the nucleic acid sequence.

In embodiment b), the isolated nucleic acid molecule also comprises a transcription terminator controlling the expression of the nucleic acid sequence.

In embodiment c), the nucleic acid sequence is also optimized for expression in a host cell, notably in a yeast or a bacterium, advantageously in a yeast.

In other embodiments, the isolated nucleic acid molecule may combine several of the features a) to c). In particular, the isolated nucleic acid molecule may be characterized in that:
d) the isolated nucleic acid molecule also comprises a promoter and a transcription terminator controlling the expression of the nucleic acid sequence (combination of a) and b));
e) the isolated nucleic acid molecule also comprises a promoter controlling the expression of the nucleic acid sequence and the nucleic acid sequence is also optimized for expression in a host cell, notably in a yeast or in a bacterium, advantageously in a yeast (combination of a) and c));
f) the isolated nucleic acid molecule also comprises a transcription terminator controlling the expression of the nucleic acid sequence and the nucleic acid sequence is also optimized for expression in a host cell, notably in a yeast or in a bacterium, advantageously in a yeast (combination of b) and c)); or
g) the isolated nucleic acid molecule also comprises a promoter and a transcription terminator controlling the expression of the nucleic acid sequence and the nucleic acid sequence is also optimized for expression in a host cell, notably in a yeast or in a bacterium, advantageously in a yeast (combination of a), b) and c)).

When the isolated nucleic acid molecule also comprises a promoter controlling the expression of the nucleic acid sequence (embodiments a), d), e) and g) above), the promoter is advantageously an exogenous promoter, in particular a yeast promoter, and preferably a promoter chosen from ADH2 (pADH2), CCW12 (pCCW12) and TEF1 (pTEF1), more preferably a promoter chosen from ADH2 (pADH2) from Saccharomyces cerevisiae, CCW12 (pCCW12) from S. cerevisiae and TEF1 (pTEF1) from S. cerevisiae, more preferably a promoter chosen from ADH2 of sequence SEQ ID NO: 20, CCW12 of sequence SEQ ID NO: 21 and TEF1 of sequence SEQ ID NO: 22.

When the isolated nucleic acid molecule also comprises a transcription terminator controlling the expression of the nucleic acid sequence (embodiments b), d), f), and g) above), the transcription terminator is advantageously an exogenous terminator, such as a yeast terminator, and preferably the RPL3 terminator (tRPL3) or the ADH1 terminator (tADH1), more preferably the RPL3 terminator of S. cerevisiae or the ADH1 terminator of S. cerevisiae, more preferably the RPL3 terminator of sequence SEQ ID NO: 23 or the ADH1 terminator of sequence SEQ ID NO: 24.

When the nucleic acid sequence is also optimized for expression in a yeast (embodiments c), e), f) and g) above), it is advantageously optimized for expression in a yeast of the genus Saccharomyces, preferably of the species Saccharomyces cerevisiae. In particular, it is advantageously chosen from SEQ ID NO: 25 to SEQ ID NO: 27.

When the nucleic acid sequence is also optimized for expression in a bacterium (embodiments c), e), f), and g) above), it is advantageously optimized for expression in a bacterium of the genera Escherichia (notably strains of the species Escherichia coli, the species most widely used for recombinant protein production) and Pseudomonas.

### Vector According to the Invention

The invention also relates to a vector comprising a nucleic acid molecule according to the invention.

The vector according to the invention may comprise any nucleic acid molecule according to the invention described above. It may be chosen from any type of vector described previously in the section concerning the vectors that may be used in the use according to the invention.

### Host Cell According to the Invention

The invention also relates to a host cell comprising a nucleic acid molecule according to the invention, or a vector according to the invention.

The host cell according to the invention may comprise any nucleic acid molecule according to the invention described above or any vector according to the invention as described above. It may also comprise any feature or combination of features described previously in the section relating to the host cells that may be used in the use according to the invention.

Methods for Producing a Polypeptide Chosen from Type III Polyketide Synthases from Ascomycete Fungi

The present invention also relates to a method for producing a polypeptide chosen from the type III polyketide synthases from ascomycete fungi as defined above.

According to one embodiment, the method for producing such a polypeptide comprises, consists essentially of, or is constituted of the following steps:
(i) the introduction of a nucleic acid molecule or a vector comprising a nucleic acid molecule as described above (nucleic acid molecule or vector used in the invention or according to the invention) into a suitable host cell in accordance with the preceding description; and
(ii) in vitro culturing of said host cell obtained in step (i) under conditions allowing the expression of said nucleic acid molecule, so as to produce said polypeptide.

According to another embodiment, the method for producing such a polypeptide comprises, consists essentially of, or is constituted at least of the step consisting of:
(ii) in vitro culturing of a host cell expressing said polypeptide, for instance a host cell as described above (host cell used in the invention or according to the invention), under conditions allowing the expression of the nucleic acid molecule contained in said host cell, so as to produce said polypeptide.

Step (i) of introducing the nucleic acid molecule or vector into a suitable host cell may be performed via any suitable method known to those skilled in the art, such as calcium phosphate transfection, transfection by liposomes comprising the nucleic acid molecule or vector to be transfected, transfection by polycationic agents, electroporation, and heat shock transfection. In particular, step (i) may be performed by transfection (via any method described above) into the host cell of a transfer plasmid comprising the sequence coding for the polypeptide chosen from type III polyketide synthases of ascomycete fungi as defined above, flanked at the 5′ and 3′ ends by sequences homologous to genomic sequences of the host cell, thus enabling homologous recombination between the transfer plasmid and the host cell genome. This method is notably applicable when the host cell is a yeast, in particular Saccharomyces, preferably of the species Saccharomyces cerevisiae, or a bacterium.

Step (ii) of in vitro culturing of the host cell is performed under conditions allowing the expression of the nucleic acid molecule contained in said host cell, so as to produce said polypeptide. These conditions vary as a function of the host cell used, and a person skilled in the art will know how to determine them on the basis of his general knowledge. In particular, when the host cell is a yeast, in particular Saccharomyces, preferably of the species Saccharomyces cerevisiae, step (ii) may be performed via any suitable method, notably as described in the section above relating to the host cells used in the use according to the invention.

The method for producing the polypeptide may also comprise at least one additional step chosen from the following steps:
(α) the recovery of host cells expressing said polypeptide and/or of the supernatant comprising the polypeptide, obtained after the culture step; and
(β) purification of the polypeptide from the host cells and/or supernatant recovered in step (α).

The optional step (α) of recovering the host cells expressing said polypeptide and/or of the supernatant comprising the polypeptide may be performed via any suitable technique known to those skilled in the art. The cells and supernatant may notably be separated by decantation or centrifugation.

The optional step (β) of purifying the polypeptide from the host cells and/or supernatant recovered in step (α) may be performed via any suitable technique known to those skilled in the art. When the polypeptide is purified from the supernatant, any liquid-phase protein purification technique may be used, for instance the various types of chromatography (gel filtration, ion exchange, by hydrophobic interaction, by affinity when the protein comprises an affinity tag, high performance liquid chromatography “HPLC”). When the polypeptide is purified from the cells or from the cells and supernatant, the purification also comprises one or more prior steps of cell lysis and optionally removal of cell debris, before the use of a liquid phase purification technique.

### Methods for Producing Phloroglucinol

As explained in the “Use” sub-section of the section covering the use according to the invention, various methods for producing phloroglucinol may be performed depending on whether they are based on the polypeptide chosen from type III polyketide synthases from ascomycete fungi, the nucleic acid molecule encoding same, the vector comprising a nucleic acid molecule encoding same or the host cell expressing same, each as defined above.

In particular, the nucleic acid molecule encoding the polypeptide chosen from the type III polyketide synthases from ascomycete fungi as described above and the vector comprising a nucleic acid molecule encoding this polypeptide can be used for transfecting a host cell, which can then be used either to produce the polypeptide (which can then produce phloroglucinol in vitro) or to produce phloroglucinol directly when it is cultured in the presence of a suitable substrate.

Thus, according to one embodiment M1, the method for producing phloroglucinol uses a host cell expressing the polypeptide chosen from the type III polyketide synthases from ascomycete fungi as defined above (for example, a host cell according to the invention), which is cultured in the presence of a substrate suitable for producing phloroglucinol. This embodiment can be subdivided into two sub-embodiments, according to whether the method comprises (embodiment M1A) or not (embodiment M1B) a preliminary step of producing the host cells.

In embodiment M1A, the invention relates to a method for producing phloroglucinol which comprises, consists essentially of, or is constituted of the following steps:
(i1) introducing a nucleic acid molecule or a vector comprising a nucleic acid molecule as described above (nucleic acid molecule or vector used in the invention or according to the invention) into a suitable host cell in accordance with the preceding description;
(ii1) placing the host cells obtained in step (i1) in contact with a suitable substrate;
(iii1) in vitro culturing of the host cell of step (ii1) under conditions allowing the expression of the nucleic acid molecule contained in said host cell, so as to produce phloroglucinol;
(iv1) optionally, recovering the phloroglucinol-comprising culture medium obtained after step (iii1); and
(vi) optionally, purification of phloroglucinol from the culture medium of step (iv1).

In embodiment M1B, the invention relates to a method for producing phloroglucinol, which comprises, consists essentially of, or is constituted of the following steps:
(ii1) placing a non-human host cell expressing the polypeptide chosen from the type III polyketide synthases from ascomycete fungi as defined above (for example a host cell according to the invention), in contact with a suitable substrate;
(iii1) in vitro culturing of the host cell of step (ii1) under conditions allowing the expression of the nucleic acid molecule contained in said host cell, so as to produce phloroglucinol;
(iv1) optionally, recovering the phloroglucinol-comprising culture medium obtained after step (iii1); and
(vi) optionally, purification of phloroglucinol from the culture medium of step (iv1).

For the purposes of methods M1A and M1B, and notably when the host cell is a yeast (in particular of the genus Saccharomyces, preferably of the species Saccharomyces cerevisiae):
step (i1) of introducing the nucleic acid molecule or vector into a suitable host cell may be performed via any method described herein in the context of step (i) of the methods for producing the polypeptide chosen from the type III polyketide synthases from ascomycete fungi as described above.
the substrate placed in contact with the host cell in step (ii1) is advantageously a carbon source. Advantageously, the carbon source is a pure carbon source or an industrial coproduct (such as molasses or green syrup, for instance from the sugar industry). Preferably, the substrate in the pure carbon source or industrial coproduct is a simple sugar, such as glucose (or dextrose), fructose, galactose, mannose, sucrose, lactose or maltose; a complex sugar, such as a monosaccharide, disaccharide or trisaccharide, or a polysaccharide such as starch; an alcohol, such as ethanol; an acid; a fatty acid and an ester derivative thereof; or a mixture of sugars, alcohols, acids and/or fatty acids or ester derivatives thereof. Preferably, the substrate is glucose or ethanol.
step (iii1) of culturing the host cell in vitro under conditions allowing the expression of the nucleic acid molecule contained in said host cell so as to produce phloroglucinol may be performed via any method described herein in the context of step (ii) of the methods for producing the polypeptide chosen from the type III polyketide synthases from ascomycete fungi as described above.
when present, step (iv1) of recovering the culture medium comprising phloroglucinol may be performed via any technique described herein for separating cells and supernatant in the context of optional step (a) of the methods for producing the polypeptide chosen from the type III polyketide synthases from ascomycete fungi as described above.
when present, step (v1) of purifying phloroglucinol from the culture medium may be performed via any suitable technique, such as liquid-liquid extraction.

Alternatively, the polypeptide chosen from the type III polyketide synthases from ascomycete fungi as described above can be used directly to produce phloroglucinol in vitro, on condition that it is brought into contact with malonyl-CoA. Specifically, phloroglucinol synthases perform the condensation of three malonyl-CoA molecules to form one phloroglucinol molecule according to the Reaction I reaction scheme described previously.

Thus, according to one embodiment M2, the phloroglucinol production method uses the polypeptide chosen from the type III polyketide synthases from ascomycete fungi as described above to produce phloroglucinol in vitro in the presence of malonyl-CoA. This embodiment can be subdivided into two sub-embodiments, according to whether the method comprises (embodiment M2A) or does not comprise (embodiment M2B) a prior step of producing the polypeptide chosen from the type III polyketide synthases from ascomycete fungi as defined above.

In embodiment M2A, the invention relates to a method for producing phloroglucinol, which comprises, consists essentially of, or is constituted of the following steps:
(i2) the production of a polypeptide chosen from the type III polyketide synthases from ascomycete fungi as defined above via one of the methods for producing polypeptides described above;
(ii2) placing the polypeptide obtained in step (i2) of the method as described above in contact with malonyl-CoA;
(iii2) incubating the mixture from step (ii2) under conditions suitable for producing phloroglucinol;
(iv2) optionally recovering the phloroglucinol-comprising reaction medium obtained after step (iii2); and
(v2) optionally, purifying phloroglucinol from the reaction medium of step (iv2).

In embodiment M2B, the invention relates to a method for producing phloroglucinol, which comprises, consists essentially of, or is constituted of the following steps:
(ii2) placing a polypeptide chosen from the type III polyketide synthases from ascomycete fungi as defined above in contact with malonyl-CoA;
(iii2) incubating the mixture from step (ii2) under conditions suitable for producing phloroglucinol;
(iv2) optionally recovering the phloroglucinol-comprising reaction medium obtained after step (iii2); and
(v2) optionally, purifying phloroglucinol from the reaction medium of step (iv2).

For the purposes of these methods M2A and M2B, and notably when the host cell is a yeast (in particular of the genus Saccharomyces, preferably of the species Saccharomyces cerevisiae):
step (i2) may be performed via any method described herein for producing a polypeptide chosen from type III polyketide synthases from ascomycete fungi.
step (ii2) consists in placing the polypeptide in contact with its substrate malonyl-CoA to allow the production of phloroglucinol. This placing in contact is performed in a medium and at a temperature which do not impair the enzymatic activity of the polypeptide. A suitable medium may notably be chosen from a sodium phosphate buffer at pH 7 (notably at a concentration of about 25-75 mM) comprising NaCl (notably at a concentration of about 5-15 mM) and bovine serum albumin (BSA, notably at a concentration of about 150-250 mg/L). A suitable temperature is in the range 20-37° C.
step (iii2) consists in incubating the polypeptide and its substrate under conditions which allow the production of phloroglucinol. These conditions involve an incubation medium and temperature which do not impair the enzymatic activity of the polypeptide, as described above for step (ii2). The incubation time is chosen as a function of the amount of substrate present in the medium. A person skilled in the art will know how to adapt the initial substrate concentration to the incubation time. It may also be possible to add substrate during the incubation period to continue phloroglucinol production.
when present, step (iv2) of recovering the reaction medium does not require any particular technique, as there are no cells to exclude or lyse prior to purification.
when present, step (v2) of purifying phloroglucinol from the culture medium may be performed via any suitable technique, such as liquid-liquid extraction.

The examples that follow are directed towards illustrating the present invention.

### EXAMPLES

### Example 1. Identification of Type III Polyketide Synthases from Ascomycete Fungi

PhlDs are type III polyketide synthases (PKS) involved in the biocatalytic synthesis of phloroglucinol from three molecules of malonyl-CoA (Zha et al., 2006). To identify new PhlDs using a sequence-based approach, the sequence of Tsukamurella pulmonis PhlD (PhlD_Tpu) was used. The latter proved to be the best phloroglucinol-producing enzyme of the 12 previously tested in S. cerevisiae, the host organism used for cloning and protein production (WO2019/002799).

A blast (Johnson et al., 2008) [NCBI] of the PhlD_Tpu protein sequence was produced against standard protein databases (non-redundant protein sequences, protein databank, Swissprot, proteins from metagenomes and patented protein sequences, versions as of Nov. 22, 2019). The sequences obtained were filtered by collecting all sequences having a percentage of identity greater than 20% relative to the input sequence. The redundant sequences were then removed (CD-HIT, sequence identity >95%), reducing the dataset to about 3320 sequences. Furthermore, sequences of 45 characterized PKSs from the literature were included if they were not present in the previously generated dataset. The sequences were finally combined using a sequence similarity network (“SSN”, Gerlt, et al., 2015), with each group corresponding to a different phylum or class. SSNs are useful for analysing sequence datasets and studying sequence-function relationships. SSN is a convenient way of visualizing relationships between protein sequences via the construction of a graph where each member of a protein family is represented by a node connected via an edge to the nodes of all other members that share a sequence similarity greater than a user-specified value. These graph networks are easy to visualize and are more readily interpretable than traditional dendrogram and phylogenetic tree approaches. This representation allows a putative function to be more readily attributed to a protein in a given cluster if a member has already been characterized, and to track evolutive relationships between clusters.

These graphs may be visualized using visualization software such as Cytoscape (Shannon et al., 2003), in which subregions of the graphs (edges and nodes) can be enlarged or manipulated iteratively, thus allowing better identification of sequence similarity at the microscopic scale of the network.

After careful cluster analysis, PKS sequences already characterized as producing phloroglucinol or other related compounds known to be produced by PKS family enzymes were identified. The majority of these enzymes are found in the plant, brown algal and actinobacterial groups. However, other enzymes were also characterized in other isolated clusters. The P. fluorescens PhlD and PhlD_Tpu sequences known to produce phloroglucinol were also identified, validating the method used.

To enhance the classification of the sequences and their interconnections, clusters of sequences from different classes were generated with an E value<10⁻¹³⁰. This allowed the recovery of two type III PKSs from brown macroalgae (Ectocarpus siliculosus-PKS1.Es and Sargassium binderi-PHLD.Sbi) already reported to produce phloroglucinol in Saccharomyces cerevisiae (WO2019/002799). The use of a stricter threshold score (E value<10⁻¹⁷⁰) allowed the separation of a cluster containing type III PKSs of the genus Tsukamurella, also known to have high phloroglucinol-producing activity in S. cerevisiae (WO2019/002799). These results once again validate the method used.

Finally, the information from the two clustering analyses with an E value<10⁻¹³⁰ and an E value<10⁻¹⁷⁰ was combined and a total of 23 sequences were selected from seven clusters and different classes to evaluate their ability to produce phloroglucinol.

### Example 2. Sequences of Three Type III Polyketide Synthases from Ascomycete Fungi Identified in Example 1

The polypeptides of amino acid sequences SEQ ID NO: 1, SEQ ID NO: 2 and SEQ ID NO: 3 are among the 23 sequences selected in Example 1, and all three are derived from ascomycete fungi.

### More Particularly:

SEQ ID NO: 1 corresponds to the amino acid sequence of a PhlD from Hirsutella minnesotensis 3608 (denoted PlhD-Hm2);
SEQ ID NO: 2 corresponds to the amino acid sequence of a PhlD from Hirsutella minnesotensis 3608 (designated P5 hD-Hm7); and
SEQ ID NO: 3 corresponds to the amino acid sequence of a PhlD from Aspergillus tanneri (denoted PlhD-At).

A multiple alignment of their amino acid sequences is shown in FIG. 1.

Their percentage of identity in pairs, and also relative to other prior art PhlDs, is also shown in Table 3 below.

TABLE 3

| | Database | | | | |
|---|---|---|---|---|---|
| | reference | Species | Hm2 | Hm1 | At |
| PHLD.Hm2 | KJZ76948.1 | Hirsutella | 100.0 | 57.5 | 52.9 |
| | | minnesotensis | | | |
| | | 3608 | | | |
| PHLD.Hm1 | KJZ69802.1 | Hirsutella | 57.5 | 100.0 | 54.4 |
| | | minnesotensis | | | |
| | | 3608 | | | |
| PHLD.At | KAA8643850.1 | Aspergillus | 52.9 | 54.4 | 100.0 |
| | | tanneri | | | |
| PKS1.Es | CBN76919.1 | Ectocarpus | 36.3 | 35.1 | 34.9 |
| | | siliculosus | | | |
| PHLD.Pf | AAY95147.1 | Pseudomonas | 21.0 | 22.1 | 20.0 |
| | | fluorescens Pf-5 | | | |
| PHLD.Tp | WP_013126955.1 | Tsukamurella | 48.8 | 50.3 | 45.5 |
| | | paurometabola | | | |
| PHLD.Tt | WP_068525790.1 | Tsukamurella | 47.1 | 48.4 | 45.9 |
| | | tyrosinosolvens | | | |
| PHLD.Tps | WP_068569959.1 | Tsukamurella | 47.7 | 52.6 | 45.5 |
| | | pseudospumae | | | |
| PHLD.Tpu | WP_068567598.1 | Tsukamurella | 49.5 | 49.4 | 46.4 |
| | | pulmonis | | | |
| PHLD.Tsp | WP_019202763.1 | Tsukamurella | 47.1 | 50.6 | 46.5 |
| | | sp. 1534 | | | |
| PHLD.Nf | WP_011211464.1 \| | Nocardia | 52.9 | 53.0 | 48.4 |
| | | farcinica | | | |
| PHLD.Mma | WP_012393954.1 \| | Mycobacterium | 54.0 | 54.3 | 48.1 |
| | | marinum | | | |
| PHLD.Mk | ZP_04749411.1 \| | Mycobacterium | 52.0 | 53.0 | 49.8 |
| | | kansasii ATCC | | | |
| | | 12478 | | | |
| PHLD.Mt | WP_031677738.1 | Mycobacterium | 43.7 | 44.7 | 41.0 |
| | | tuberculosis | | | |
| PHLD.Gh | WP_066172590.1 | Gordonia | 46.0 | 47.9 | 40.7 |
| | | hydrophobica | | | |
| PHLD.Sbi | ADK13089.1 | Sargassum | 38.7 | 35.6 | 33.8 |
| | | binderi | | | |
| PHLD-1.Aa | SEQ ID NO: 3 of | Aureococcus | 37.8 | 38.3 | 35.2 |
| | WO2019/002798 | anophagefferens | | | |

Table 3 above shows that the three sequences newly identified as possible phloroglucinol synthases have only weak sequence identity with the sequences of previously identified phloroglucinol synthases, and belong to a different kingdom (fungi versus bacteria or eukaryotic algae).

### Example 3. Ability of Type III Polyketide Synthases from Ascomycete Fungi to Produce Phloroglucinol in Yeast

The three putative phloroglucinol synthases previously identified (SEQ ID NO: 1, SEQ ID NO: 2 and SEQ ID NO: 3) were tested for their ability to produce phloroglucinol when expressed by yeast of the Saccharomyces cerevisiae species. Their activity was also compared with that of prior art PHLDs.

### Materials and Methods

### Yeast Cells

Cells from the yeast S. cerevisiae CEN.PK2-1D (see Entian K. D. and Kötter P., 2007, commercially available notably from Euroscarf) were used.

Preparation of S. cerevisiae CEN.PK2-1 D Cell Clones Expressing the Three Putative Phloroglucinol Synthases or Phloroglucinol Synthases of the Prior Art

The nucleic acid sequences SEQ ID NO: 25 to 27 coding respectively for the three putative phloroglucinol synthases previously identified (SEQ ID NO: 1, SEQ ID NO: 2 and SEQ ID NO: 3) were modified by adding to their 5′ and 3′ ends, respectively, sequences homologous to the desired site of integration into the genome of S. cerevisiae CEN.PK2-1D yeast cells, resulting in sequences SEQ ID NO: 28 to 30. For phloroglucinol synthases PHLD.Pf, PHLD.Tsp and PHLD.Tt, sequences SEQ ID NO: 31 to 33, which comprise sequences homologous to the desired site of integration into the genome, were used.

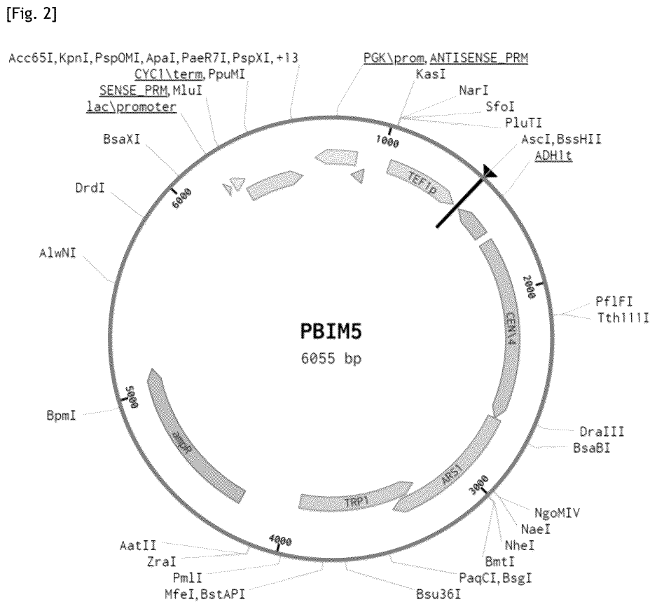

The sequences SEQ ID NO: 28 to 33 were then cloned into a plasmid called “PBIM5” or “YCplac22_PGK_CYC_TEF_ADH” (derived from the commercial plasmid YCplac22 by adding the pair (PGK promoter and CYC terminator, see Gietz R. D. et al., 1988) and also the pair (TEF1 promoter and ADH1 terminator), see FIG. 2), under the control of the TEF1 promoter and the ADH1 terminator. The sequences SEQ ID NO: 28 to 33 were then integrated into the genome of S. cerevisiae CEN.PK2-1D yeast cells by homologous recombination in vivo, and the cloned sequences verified by Sanger sequencing.

### 96-Well Plate Culture for Phloroglucinol Production

Each well was filled with 500 μl of complete synthetic Trp_drop out medium sold by Formedium.

A single cell clone obtained as described above was collected and inoculated into a well.

The plate was incubated at 30° C. for about 20 hours at 800 rpm in a vibrating incubator. The following day, a new 96-well plate filled with 500 μl of complete synthetic medium was inoculated with 10 μl of culture. Three individual plates were inoculated for 24-, 48- and 72-hour sampling.

### Quantification of Phloroglucinol Production by Colorimetric Test

Every 24 hours, each plate was removed from the incubator and measurements were taken as described below:
Firstly, the absorbance was measured in the microtitre plate at 600 nm using the Tecan plate reader. For this, 20 μl of culture were diluted with 180 μl of MilliQ water.
Next, the 96-well plate was centrifuged at 3700 rpm for 10 minutes and the supernatant was used for the colorimetric assay.
150 μl of 500 mg/L 4-hydroxy-3-methoxycinnamaldehyde in HCl/EtOH v:v (1:3) were added to the sample and to the phloroglucinol standard. Incubation was performed at room temperature for 30 min.

### Results

The amounts of phloroglucinol produced in the test used by the yeasts expressing the various PhlDs are shown in Table 4 below.

TABLE 4

| PhlD | Phloroglucinol production after 72 h |
|---|---|
| PHLD.Hm2 | 54.1 mg/L |
| PHLD.Hm1 | 11.8 mg/L |
| PHLD.At | 26.8 mg/L |
| PHLD.Pf | No phloroglucinol production |
| PhlD.Tsp | 47.5 mg/L |
| PhlD.Tt | 37.9 mg/L |

Table 4 shows that the three putative phloroglucinol synthases previously identified (SEQ ID NO: 1, SEQ ID NO: 2 and SEQ ID NO: 3), although derived from ascomycete fungi, are capable of producing phloroglucinol when expressed in a yeast.

### BIBLIOGRAPHIC REFERENCES

Achkar J. et al., (2005) “Biosynthesis of phloroglucinol” J. Am. Chem. Soc. 48, 443-5332, 5333.
Entian K. D. and Kötter P. Yeast Genetic Strain and Plasmid Collections. Methods in Microbiology (2007) 36: 629-666.
Gerlt, J. A. et al. Enzyme Function Initiative-Enzyme Similarity Tool (EFI-EST): A web tool for generating protein sequence similarity networks. Biochim. Biophys. Acta BBA—Proteins Proteomics 1854, 1019-1037 (2015).
Gietz R. D., Sugino A. New yeast Escherichia coli shuttle vectors constructed with in vitro mutagenized yeast genes lacking six-base pair restriction sites. Gene 74: 527-534, 1988.
Johnson, M. et al. NCBI BLAST: a better web interface. Nucleic Acids Res. 36, W5-W9 (2008).
Meslet-Cladière L., Delage L., Leroux C. J., Goulitquer S., Leblanc C., Creis E., Gall E. A., Stiger-Pouvreau V., Czjzek M., and Potin P. (2013) “Structure/function analysis of a type III polyketide synthase in the brown alga Ectocarpus siliculosus reveals a biochemical pathway in phlorotannin monomer biosynthesis.” Plant Cell. 48, 443-3089, 3103.
Needleman and Wunsch. “A General Method Applicable to The Search For Similarities in the Amino Acid Sequence of Two Proteins”, J. Mol. Biol. 48, 443-453, 1970.
Shannon, P. Cytoscape: A Software Environment for Integrated Models of Biomolecular Interaction Networks. Genome Res. 13, 2498-2504 (2003).
Zha W., Rubin-Pitel S. B. and Zhao H. (2006) “Characterization of the substrate specificity of PHLD, a type III polyketide synthase from Pseudomonas fluorescens.” J. Biol. Chem. 48, 443-32036, 32047.
WO2013/045510
WO2019/002798
WO2019/002799
WO2020/024917A1

1.-16. (canceled)

17. A method for producing phloroglucinol comprising the step of:
using (i) a polypeptide selected from the group consisting of type III polyketide synthases from ascomycete fungi, (ii) a nucleic acid molecule encoding the polypeptide selected from the group consisting of type III polyketide synthases from ascomycete fungi, (iii) a vector comprising a nucleic acid molecule encoding the polypeptide selected from the group consisting of type III polyketide synthases from ascomycete fungi or (iv) a non-human host cell expressing the polypeptide selected from the group consisting of type III polyketide synthases from ascomycete fungi, to produce phloroglucinol,
wherein the polypeptide comprises an amino acid sequence having at least 70% identity with a sequence selected from the group consisting of SEQ ID NO: 1, SEQ ID NO: 2 and SEQ ID NO: 3.

18. The method according to claim 17, wherein the polypeptide is selected from the group consisting of type III polyketide synthases from fungi of the families Ophiocordycipitaceae and Trichocomaceae.

19. The method according to claim 17, wherein the polypeptide comprises an amino acid sequence having at least 80% identity with a sequence selected from the group consisting of SEQ ID NO: 1, SEQ ID NO: 2 and SEQ ID NO: 3.

20. The method according to claim 17, wherein the polypeptide further comprises at least one fragment selected from the group consisting of:
(a) LVTSTGFX₁APGVDV, in which X₁ is selected from the group consisting of apolar amino acids (SEQ ID NO: 4);
(b)
(SEQ ID NO: 5)
VNFMGCAAAMNGLR;
(c)
(SEQ ID NO: 6)
CCVELSSVNAV;
(d) ISSLFADGX₂AAMVVGS, in which X₂ is selected from the group consisting of apolar or neutral polar amino acids (SEQ ID NO: 7);
(e)
(SEQ ID NO: 8)
WAIHPGGPKIIEES;
(f)
(SEQ ID NO: 9)
GNMLSASLPFVL;
and
(g) AFSFAPGX₃TVEG, in which X₃ is selected from the group consisting of apolar amino acids (SEQ ID NO: 10).

21. The method according to claim 20, wherein the polypeptide further comprises at least one fragment selected from the group consisting of:
(a) LVTSTGFX₁APGVDV, in which X₁ is selected from the group consisting of aliphatic apolar amino acids (SEQ ID NO: 11);
(b)
(SEQ ID NO: 5)
VNFMGCAAAMNGLR;
(c)
(SEQ ID NO: 6)
CCVELSSVNAV;
(d) ISSLFADGX₂AAMVVGS, in which X₂ is selected from the group consisting of sulfur-based apolar or neutral polar amino acids (SEQ ID NO: 12);
(e)
(SEQ ID NO: 8)
WAIHPGGPKIIEES;
(f) GNMLSASLPFVL (SEQ ID NO: 9); and
(g) AFSFAPGX₃TVEG, in which X₃ is selected from the group consisting of aliphatic apolar amino acids (SEQ ID NO: 13).

22. The method according to claim 21, wherein the polypeptide further comprises at least one fragment selected from the group consisting of:
(a) LVTSTGFX₁APGVDV, in which X₁ is selected from valine and isoleucine (SEQ ID NO: 14);
(b)
(SEQ ID NO: 5)
VNFMGCAAAMNGLR;
(c)
(SEQ ID NO: 6)
CCVELSSVNAV;
(d) ISSLFADGX₂AAMVVGS, in which X₂ is selected from cysteine and serine (SEQ ID NO: 15);
(e)
(SEQ ID NO: 8)
WAIHPGGPKIIEES;
(f) GNMLSASLPFVL (SEQ ID NO: 9); and
(g) AFSFAPGX₃TVEG, in which X₃ is selected from valine and isoleucine (SEQ ID NO: 21).

23. The method according to claim 17, wherein the polypeptide comprises an amino acid sequence selected from the group consisting of SEQ ID NO: 1, SEQ ID NO: 2 and SEQ ID NO: 3.

24. An isolated nucleic acid molecule comprising a nucleic acid sequence encoding a polypeptide selected from the group consisting of type III polyketide synthases from ascomycete fungi,
wherein the polypeptide comprises an amino acid sequence having at least 70% identity with a sequence selected from the group consisting of SEQ ID NO: 1, SEQ ID NO: 2 and SEQ ID NO: 3, and
wherein:
(a) the isolated nucleic acid molecule further comprises a promoter controlling expression of the nucleic acid sequence; or
(b) the isolated nucleic acid molecule further comprises a transcription terminator controlling the expression of the nucleic acid sequence; or
(c) the nucleic acid sequence is optimized for expression in a host cell; or
(d) any combination of (a) to (c).

25. The isolated nucleic acid molecule according to claim 24, wherein, when the isolated nucleic acid molecule further comprises the promoter, the promoter is an exogenous promoter.

26. The isolated nucleic acid molecule according to claim 24, wherein, when the isolated nucleic acid molecule further comprises the transcription terminator, the transcription terminator is an exogenous terminator.

27. The isolated nucleic acid molecule according to claim 24, wherein the nucleic acid sequence is optimized for expression in a yeast.

28. A non-human host cell comprising the isolated nucleic acid molecule according to claim 24.

29. The non-human host cell according to claim 28, wherein the host cell is a yeast.

30. A vector comprising the isolated nucleic acid molecule according to claim 24.

31. A non-human host cell comprising the vector according to claim 30.

32. The non-human host cell according to claim 31, wherein the host cell is a yeast.

33. A method for producing phloroglucinol comprising the following steps:
(ii1) placing a non-human host cell expressing the polypeptide selected from the group consisting of type III polyketide synthases from ascomycete fungi, wherein the polypeptide comprises an amino acid sequence having at least 70% identity with a sequence selected from the group consisting of SEQ ID NO: 1, SEQ ID NO: 2 and SEQ ID NO: 3, in contact with a suitable substrate;
(iii1) in vitro culturing of the non-human host cell of step (ii1) under conditions allowing expression of a nucleic acid molecule contained in the non-human host cell, so as to produce phloroglucinol;
(iv1) optionally, recovering a phloroglucinol-comprising culture medium obtained after step (iii1); and
(v1) optionally, purifying phloroglucinol from the phloroglucinol-comprising culture medium of step (iv1).

34. A method for producing phloroglucinol comprising the following steps:
(ii2) placing a polypeptide selected from the group consisting of type III polyketide synthases from ascomycete fungi, wherein the polypeptide comprises an amino acid sequence having at least 70% identity with a sequence selected from the group consisting of SEQ ID NO: 1, SEQ ID NO: 2 and SEQ ID NO: 3 in contact with malonyl-CoA;
(iii2) incubating a mixture from step (ii2) under conditions suitable for producing phloroglucinol;
(iv2) optionally recovering a phloroglucinol-comprising reaction medium obtained after step (iii2); and
(v2) optionally, purifying phloroglucinol from the phloroglucinol-comprising reaction medium of step (iv2).

35. The method according to claim 18, wherein the polypeptide is selected from the group consisting of type III polyketide synthases from fungi from the species Hirsutella minnesotensis 3608 and Aspergillus tanneri.

36. The isolated nucleic acid molecule according to claim 25, wherein the promoter is a yeast promoter.

37. The isolated nucleic acid molecule according to claim 27, wherein the nucleic acid sequence is selected from the group consisting of SEQ ID NO: 25 to SEQ ID NO: 27.

38. The non-human host cell according to claim 29, wherein the host cell is of the Saccharomyces cerevisiae species.

39. The non-human host cell according to claim 32, wherein said host cell is of the Saccharomyces cerevisiae species.
